SEQUENCE LISTING (12 sequences)

SEQ ID NO: 1 - DNA, 30 bp, Artificial Sequence
  DNA probe 1
gcgcgctggctggggcgccgcgcccgcgct

SEQ ID NO: 2 - DNA, 30 bp, Artificial Sequence
  DNA target 1
agcgcgggcgcggcgccccagccagcgcgc

SEQ ID NO: 3 - DNA, 30 bp, Artificial Sequence; identical to SEQ ID NO: 2
  methyl DNA target 1 (C5)

SEQ ID NO: 4 - DNA, 30 bp, Artificial Sequence; identical to SEQ ID NO: 2
  methyl DNA target 2 (C14)

SEQ ID NO: 5 - DNA, 30 bp, Artificial Sequence; identical to SEQ ID NO: 2
  methyl DNA target 3 (C26)

SEQ ID NO: 6 - DNA, 33 bp, Artificial Sequence
  DNA probe 2
cgaaatgatcccatccagctgcgcgttgaccgc

SEQ ID NO: 7 - DNA, 33 bp, Artificial Sequence
  DNA target 2
gcggtcaacgcgcagctggatgggatcatttcg

SEQ ID NO: 8 - DNA, 33 bp, Artificial Sequence; identical to SEQ ID NO: 7
  methyl DNA target 4 (C2)

SEQ ID NO: 9 - DNA, 33 bp, Artificial Sequence; identical to SEQ ID NO: 7
  methyl DNA target 5 (C2,16)

SEQ ID NO: 10 - DNA, 33 bp, Artificial Sequence; identical to SEQ ID NO: 7
  methyl DNA target 6 (C2,16,32)

SEQ ID NO: 11 - DNA, 48 bp, Artificial Sequence
  methyl DNA target 7 (C39)
gcggtcaacgcgcagctggatgggatcatttcggacttcgaaggtggg

SEQ ID NO: 12 - DNA, 33 bp, Artificial Sequence
  N-DNA probe
nganatnatnccntcnagctgcgcgttgaccgc

(19) United States
(12) Patent Application Publication
(10) Pub. No.: US 2017/0016051 A1
(43) Pub. Date: Jan. 19, 2017

(54) DNA Methylation Detection

(71) Applicant: OriZhan Bioscience Limited, Taipei City (TW)

(72) Inventors: Hardy Wai-Hong CHAN, Redwood City, CA (US); Yuh-Shyong YANG, Hsinchu (TW); Wen-Yih CHEN, Taoyuan City (TW); Chang-Wei FU, Pingtung County (TW)

(21) Appl. No.: 15/210,515

(22) Filed: Jul. 14, 2016

Related U.S. Application Data
(60) Provisional application No. 62/192,987, filed on Jul. 15, 2015.

(51) Int. Cl. C12Q 1/68; G01N 27/414

(52) U.S. Cl. CPC C12Q 1/6825; G01N 27/4145

(57) ABSTRACT

The present invention relates to a method for detecting methylation of a target oligonucleotide molecule. The method comprises obtaining an electrical change occurring due to the binding of an electrically charged methylation detecting molecule and the target oligonucleotide molecule; wherein the electrically charged methylation detecting molecule has affinity to a methylated cytosine nucleotide. The invention improves the detection sensitivity and accuracy of the oligonucleotide methylation detection.

### FIELD OF THE INVENTION

This invention relates to a molecular detection technique. More particularly, the invention relates to DNA methylation detection.

### BACKGROUND OF THE INVENTION

Molecular detection plays an important role in clinical diagnosis and molecular biology research. For example, DNA methylation detection is regarded as a key detection in several applications. The methylation of the 5′ carbon of cytosine (also known as methylated cytosine or 5-methylcytosine) in DNA is an epigenetic modification that regulates gene expression and plays crucial roles in embryonic development, and is thought to be involved in cancer, genomic imprinting, cellular differentiation, and Alzheimer's disease (Kurita, Ryoji, and Osamu Niwa. “DNA methylation analysis triggered by bulge specific immuno-recognition.” Analytical chemistry 84.17 (2012): 7533-7538).

Several systems have been developed to perform DNA methylation detection for detecting and/or identifying the number and/or position of 5-methylcytosine in a DNA molecule. The major approach for DNA methylation detection is a bisulfite conversion method (Lister, Ryan, et al. “Human DNA methylomes at base resolution show widespread epigenomic differences.” Nature 462.7271 (2009): 315-322). In the method, a DNA molecule to be tested is treated with bisulfite to convert cytosine nucleotides to uridine nucleotides, leaving 5-methylcytosine nucleotides unconverted. The converted uridine nucleotides are further converted to thymine nucleotides in the subsequent replication of polymerase chain reaction. On the other hand, the unconverted 5-methylcytosine nucleotides remain cytosine nucleotides in the replication. For distinguishing the converted thymine nucleotides from unconverted cytosine nucleotides, a gene sequencing or microarray process is applied. For example, the whole genome shotgun bisulfite sequencing (WGSGS) is utilized to analyze the whole gene sequence after bisulfite conversion, and the methylation pattern in a specific situation can be obtained with single-base resolution. However, the sequencing is laborious and costly. In order to remedy the defects, a method of reduced representation bisulfite sequencing (RRBS) coupled with restriction enzymes was developed to eliminate the unnecessary fragments, but the cost is still unsatisfactory. Another approach is using the microarray process, which is able to analyze the methylation pattern of a specific fragment in a fast and high-throughput manner, and is widely applied in drug screening Nevertheless, the microarray process fails to determine the number and position of the 5-methylcytosine nucleotides.

An isoschizomer method for DNA methylation detection has also been developed. Isoschizomers are pairs of restriction enzymes specific to the same recognition site. One of the common isoschizomers is a methylation-sensitive enzyme that recognizes only unmethylated cytosine nucleotides and does not recognize methylated cytosine nucleotides. Another common isoschizomers is a methylation-dependent enzyme that recognizes only methylated cytosine nucleotides and does not recognize un-methylated cytosine nucleotides. By incorporating different kinds of isoschizomers, the 5-methylcytosine nucleotides can be detected. Unfortunately, application of the method is highly limited due to the inherent property of the isoschizomers, and only the 5-methylcytosine nucleotides located in the recognition site of a specific pair of isoschizomers can be detected.

Another approach for DNA methylation detection is an affinity method. Antibodies or proteins with specific affinity to the 5-methylcytosine nucleotides are utilized for capturing a fragment containing 5-methylcytosine nucleotides, and a gene sequencing or microarray process is applied subsequently to analyze the captured fragment. Examples of affinity methods are methylated DNA immunoprecipitation (MeDIP) using the antibodies (Weber, Michael, et al. “Chromosome-wide and promoter-specific analyses identify sites of differential DNA methylation in normal and transformed human cells.” Nature genetics 37.8 (2005): 853-862) or methylated CGI recovery assay (MIRA) using methyl binding domain proteins (MBDs) (Cross, Sally H., et al. “Purification of CpG islands using a methylated DNA binding column.” Nature genetics 6.3 (1994): 236-244; Kurita, Ryoji, and Osamu Niwa. “DNA methylation analysis triggered by bulge specific immuno-recognition.” Analytical chemistry 84.17 (2012): 7533-7538). In the affinity method, the fragment is provided by a manner such as sonication and the single-stranded fragment is also provided if necessary. The antibody or protein is first used to capture the fragment containing 5-methylcytosine nucleotides. After being separated from the antibody or protein, the captured fragment is analyzed by gene sequencing or microarray. Because the sequencing or microarray process is needed, the defects resulting from the process as mentioned above cannot be avoided.

### SUMMARY OF THE INVENTION

In order to improve DNA methylation detection sensitivity and accuracy, a quick and convenient detection method is provided.

The invention is to provide a method for detecting methylation of a target oligonucleotide molecule, comprising obtaining an electrical change occurring due to the binding of an electrically charged methylation detecting molecule and the target oligonucleotide molecule; wherein the electrically charged methylation detecting molecule has affinity to a methylated cytosine nucleotide.

The present invention is also to provide a kit for detecting methylation of a target oligonucleotide molecule comprising:
an electrically charged methylation detecting molecule having affinity to a methylated cytosine nucleotide; and
an electrical change detecting element for detecting an electrical change.

The present invention is described in detail in the following sections. Other characteristics, purposes and advantages of the present invention can be found in the detailed description and claims.

### BRIEF DESCRIPTION OF THE DRAWINGS

FIG. 1 shows a schematic drawing of a method for detecting methylation of a target oligonucleotide molecule in one embodiment of the invention.

FIG. 2 shows one preferred embodiment of the electrically neutral nucleotide according to the invention.

FIG. 3 shows I_{d}-V_{g} curve of SEPT9 DNA probe 1 with SEPT9 methyl DNA target 1 (C5). “▪” indicates the electrical signal detected in 10 mM bis-tris propane buffer; “” indicates the electrical signal induced by SEPT9 methyl DNA target 1; “▴” indicates the electrical signal induced by 1 μg/mL anti-methylcytosine antibody.

FIG. 4 shows I_{d}-V_{g} curve of SEPT9 DNA probe 1 with SEPT9 methyl DNA target 2 (C14). “▪” indicates the electrical signal detected in 10 mM bis-tris propane buffer; “” indicates the electrical signal induced by SEPT9 methyl DNA target 2; “▴” indicates the electrical signal induced by 1 μg/mL anti-methylcytosine antibody.

FIG. 5 shows I_{d}-V_{g} curve of SEPT9 DNA probe 1 with SEPT9 methyl DNA target 3 (C26). “▪” indicates the electrical signal detected in 10 mM bis-tris propane buffer; “” indicates the electrical signal induced by SEPT9 methyl DNA target 3; “▴” indicates the electrical signal induced by 1 μg/mL anti-methylcytosine antibody.

FIG. 6 shows the threshold voltage shift of nanowire FET (NWFET) induced by target DNA (left column) and antibody (right column)

FIG. 7 shows the threshold voltage shift ratio of antibody to target DNA.

FIG. 8 shows I_{d}-V_{g} curve of SEPT9 DNA probe 2 with SEPT9 methyl DNA target 6 (C2,16,32). “▪” indicates the electrical signal detected in 10 mM Bis-Tris Propane buffer; “” indicates the electrical signal induced by SEPT9 methyl DNA target 6; “▴” indicates the electrical signal induced by 0.25 μg/mL anti-methylcytosine antibody.

FIG. 9 shows the threshold voltage shift of NWFET induced by target DNA (left column) and antibody (right column). 1× means the target oligonucleotide molecule has one methylated cytosine nucleotide. 2× means the target oligonucleotide molecule has two methylated cytosine nucleotides. 3× means the target oligonucleotide molecule has three methylated cytosine nucleotides.

FIG. 10 shows the threshold voltage shift ratio of antibody to target oligonucleotide molecule.

FIG. 11 shows I_{d}-V_{g} curve of SEPT9 DNA probe 2 with SEPT9 methyl DNA target 7.

FIG. 12 shows I_{d}-V_{g} curve of partially neutral single-stranded oligonucleotide (N-DNA) probe binding with SEPT9 methyl DNA target 6 (C2, C16, C32) and antibody. “▪” indicates the electrical signal detected in 10 mM bis-tris propane buffer; “” indicates the electrical signal induced by SEPT9 methyl DNA target 6; “▴” indicates the electrical signal induced by 0.1 μg/mL anti-methylcytosine antibody.

### DETAILED DESCRIPTION OF THE INVENTION

The invention is to provide a method for detecting methylation of a target oligonucleotide molecule, comprising obtaining an electrical change occurring due to the binding of an electrically charged methylation detecting molecule and the target oligonucleotide molecule; wherein the electrically charged methylation detecting molecule has affinity to a methylated cytosine nucleotide.

As used herein, the term “an oligonucleotide” or “an oligonucleotide molecule” refers to an oligomer of nucleotide. The term “nucleotide” refers to an organic molecule composed of a nitrogenous base, a sugar, and one or more phosphate groups; preferably one phosphate group. The nitrogenous base includes a derivative of purine or pyrimidine. The purine includes substituted or unsubstituted adenine and substituted or unsubstituted guanine; the pyrimidine includes substituted or unsubstituted thymine, substituted or unsubstituted cytosine and substituted or unsubstituted uracil. The sugar is preferably a five-carbon sugar, more preferably substituted or unsubstituted ribose or substituted or unsubstituted deoxyribose. The phosphate groups form bonds with the 2, 3, or 5-carbon of the sugar; preferably, with the 5-carbon site. For forming the oligonucleotide, the sugar of one nucleotide is joined to the adjacent sugar by a phosphodiester bridge. Preferably, the oligonucleotide is DNA or RNA; more preferably, DNA.

As used herein, the term “a target oligonucleotide molecule” refers to a naturally occurring or artificial molecule. In another aspect, the target oligonucleotide molecule is purified or mixed with other contents. In one preferred embodiment of the invention, the methylation pattern of the target oligonucleotide molecule is different in a normal condition and in an abnormal condition, such as a disease. In another preferred embodiment of the invention, the methylation pattern of the target oligonucleotide molecule is different in different cell types.

As used herein, the term “methylation of a target oligonucleotide molecule” refers to methylated nucleotides in a target oligonucleotide molecule. Particularly, the methylated nucleotide refers to a methylated cytosine nucleotide. As used herein, the methylated cytosine nucleotide has a methyl substitution in the 5′ carbon of cytosine, and is also known as 5-methylcytosine nucleotide. When the target oligonucleotide molecule comprises a methylated cytosine nucleotide, the target oligonucleotide molecule has a target sequence with at least one methylated cytosine nucleotide.

As used herein, the term “detecting methylation of a target oligonucleotide molecule” refers to discovering or determining a profile or pattern of methylation of a target oligonucleotide molecule, including but not limited to the position, number or percentage of methylated nucleotides in the target oligonucleotide molecule.

As used herein, the term “an electrically charged methylation detecting molecule” refers to a molecule that has affinity to a methylated cytosine nucleotide, and carries electrical charges. Preferably, the electrically charged methylation detecting molecule is a protein that specifically binds to a methylated cytosine nucleotide. In one preferred embodiment of the invention, the electrically charged methylation detecting molecule is selected from the group consisting of an anti-methylcytosine antibody, a methyl binding domain protein and a restriction enzyme. The anti-methylcytosine antibody is a monoclonal antibody or a polyclonal antibody. In one embodiment of the invention, the anti-methylcytosine antibody recognizes a methylated cytosine nucleotide in a single-stranded oligonucleotide molecule, and in another embodiment of the invention, the anti-methylcytosine antibody recognizes a methylated cytosine nucleotide in a double-stranded oligonucleotide molecule. In another aspect, in one embodiment of the invention, the methyl binding domain protein binds to a methylated cytosine nucleotide in a single-stranded oligonucleotide molecule, and in another embodiment of the invention, the methyl binding domain protein binds to a methylated cytosine nucleotide in a double-stranded oligonucleotide molecule. Examples of the methyl binding domain protein include methy-CpG-binding domain (MBD) 1, MBD2, MBD3, and MBD4 protein. In still another aspect, in one embodiment of the invention, the restriction enzyme recognizes a methylated cytosine nucleotide in a single-stranded oligonucleotide molecule, and in another embodiment of the invention, the restriction enzyme recognizes a methylated cytosine nucleotide in a double-stranded oligonucleotide molecule. Examples of the restriction enzymes include HpaII, MspI, SmaI, and XmaI.

According to the invention, if a methylated cytosine nucleotide is present in the target oligonucleotide molecule, the electrically charged methylation detecting molecule binds to the methylated cytosine nucleotide of the target oligonucleotide molecule. Because the electrically charged methylation detecting molecule carries electrical charges, an electrical change occurs due to the binding of the electrically charged methylation detecting molecule and the target oligonucleotide molecule by introducing the electrical charges of the electrically charged methylation detection molecule into a complex formed by the electrically charged methylation detection molecule and the target oligonucleotide molecule. On the other hand, if a methylated cytosine nucleotide is absent from the target oligonucleotide molecule, the electrically charged methylation detecting molecule fails to bind to the methylated cytosine nucleotide of the target oligonucleotide molecule. Therefore, the electrical environment is unchanged, and no electrical change occurs. By monitoring of the electrical change, the methylation of the target oligonucleotide molecule is detected, and the profile or pattern of methylation of the target oligonucleotide molecule is determined.

The electrical change according to the invention includes but is not limited to increase of electrical charges. The electrical change can be detected as an electrical signal. The electrical signal includes but is not limited to changes of electric conductivity, electric field, electric capacitance, electric current, electron, or electron hole. In one preferred embodiment of the invention, the electrical change is a threshold voltage shift change.

Preferably, the electrical change is detected by an electrical change detecting element. The electrical change detecting element is applied for detecting whether the electrical change occurs due to the binding of the electrically charged methylation detecting molecule and the target oligonucleotide molecule. Preferably, the electrical change detecting element is a field-effect transistor or a surface plasmon resonance.

In one preferred embodiment of the invention, the method for detecting methylation of the target oligonucleotide molecule comprises:
capturing a single strand of the target oligonucleotide molecule by a recognizing single-stranded oligonucleotide molecule to form a duplex according to base complementarity;
providing the electrically charged methylation detecting molecule; and
binding the duplex with the electrically charged methylation detecting molecule and detecting the electrical change due to the binding.

The target oligonucleotide molecule can be a single-stranded molecule or a double-stranded molecule. The manner of obtaining the single strand of the double-stranded target oligonucleotide molecule can be, for example, heating or changing ion strength of the environment of the double-stranded target oligonucleotide molecule.

As used herein, the term “a recognizing single-stranded oligonucleotide molecule” refers to a single-stranded oligonucleotide molecule able to form a duplex with the target oligonucleotide molecule according to base complementarity. In other words, the recognizing single-stranded oligonucleotide molecule acts as a probe to hybridize the target oligonucleotide molecule. The duplex preferably refers to a double-stranded structure, and one strand is the single strand of the target oligonucleotide molecule and the other strand is the recognizing single-stranded oligonucleotide molecule. Preferably, the recognizing single-stranded oligonucleotide molecule has a sequence matched to the target sequence; more preferably, has a sequence perfectly matched to the target sequence. By forming the duplex, the target oligonucleotide molecule can be captured from a mixture in a sample. The capturing step also refers to a purification step of specifically selecting the target oligonucleotide molecule and presenting the target oligonucleotide molecule in the duplex. Consequently, the electrically charged methylation detecting molecule binds to the duplex, especially the methylated cytosine nucleotide of the target nucleotide molecule in the duplex, and the electrical change occurring due to the binding is obtained.

The sample according to the invention is derived from a naturally occurring origin or derived from artificial manipulation. Preferably, the sample is derived from a naturally occurring origin such as an extract, body fluid, tissue biopsy, liquid biopsy, or cell culture. In another aspect, the sample is processed according to the reaction of detection. For example, the pH value or ion strength of the sample may be adjusted.

Preferably, the duplex formed by the single strand of the target oligonucleotide molecule and the recognizing single-stranded oligonucleotide molecule comprises a bulge and the methylated cytosine nucleotide is disposed in the bulge. The sequence of the recognizing single-stranded oligonucleotide molecule is preferably to be designed to form the bulge with the single strand of the target oligonucleotide molecule. The bulge allows the methylated cytosine nucleotide to be presented well to the electrically charged methylation detecting molecule in the three-dimensional structure of the duplex.

The recognizing single-stranded oligonucleotide molecule according to the invention may be presented in a solution or attached to a solid surface. Preferably, the recognizing single-stranded oligonucleotide molecule is attached to a solid surface or the recognizing single-stranded oligonucleotide molecule is spaced apart from the solid surface by a distance.

As used herein, the term “solid surface” refers to a solid support including but not limited to a polymer, paper, fabric, or glass. Preferably, the solid surface to be employed varies depending on the electrical change detecting element. For example, when the method adopts a field-effect transistor to detect the electrical change, the solid surface is a transistor surface of the field-effect transistor; when the method adopts a surface plasmon resonance, the solid surface is a metal surface of a surface plasmon resonance.

In a preferred embodiment of the invention, the material of the solid surface is silicon; preferably polycrystalline silicon or single crystalline silicon; more preferably polycrystalline silicon. Polycrystalline silicon is cheaper than single crystalline silicon, but because the polycrystalline has more grain boundary, a defect usually occurs in the grain boundary that hinders electron transduction. Such phenomenon makes the solid surface uneven and quantification difficult. Furthermore, ions may penetrate into the grain boundary of the polycrystalline and cause detection failure in solution. In addition, polycrystalline silicon is not stable in air. The abovementioned drawbacks, however, would not interfere with the function of the method according to the invention.

The manner of attaching the recognizing single-stranded oligonucleotide molecule and the solid surface depends on the material of the solid surface and the type of recognizing single-stranded oligonucleotide molecule. In one embodiment of the invention, the recognizing single-stranded oligonucleotide molecule links to the solid surface through a covalent bond. Examples of the covalent bond include but are not limited to the following methods, depending on the solid surface chemistry and the modification of the oligonucleotide. In one embodiment of the invention, when silicon oxide is used as the solid surface, the solid surface is modified by using (3-Aminopropyl)triethoxysilane (APTES). The silicon atom in the molecule of APTES performs a covalent bond with the oxygen atom of the hydroxyl group and it converts the surface's silanol groups (SiOH) to amines; then the 5′-amino group of recognizing single-stranded oligonucleotide molecule is covalently bonded with the solid surface amines group by glutaraldehyde (Roey Elnathan, Moria Kwiat, Alexander Pevzner, Yoni Engel, Larisa Burstein Artium Khatchtourints, Amir Lichtenstein, Raisa Kantaev, and Fernando Patolsky, Biorecognition Layer Engineering: Overcoming Screening Limitations of Nanowire-Based FET Devices, Nano letters, 2012, 12, 5245-5254). In another embodiment of the invention, the solid surface is modified into self-assembling monolayer molecules with different functional groups for covalently linking to different functional groups of the recognizing single-stranded oligonucleotide molecule by various chemical reactions (Srivatsa Venkatasubbarao, Microarrays—status and prospects, TRENDS in Biotechnology Vol. 22 No. 12 December 2004; Ki Su Kim, Hyun-Seung Lee, Jeong-A Yang, Moon-Ho Jo and Sei Kwang Hahn, The fabrication, characterization and application of aptamer-functionalized Si-nanowire FET biosensors, Nanotechnology 20 (2009)).

In another preferred embodiment of the invention, the recognizing single-stranded oligonucleotide molecule is spaced apart from the solid surface by a distance. Since the electrical change detecting element is applied for detecting the electrical changedue to the binding of the electrically charged methylation detecting molecule and the target oligonucleotide molecule, the recognizing single-stranded oligonucleotide molecule is not necessary to directly bind to the solid surface, provided that the distance between the recognizing single-stranded oligonucleotide molecule and the solid surface is short enough to allow the electrical change detecting element to detect the electrical change. Preferably, the distance between the solid surface and the recognizing single-stranded oligonucleotide molecule is about 0 to about 10 nm; more preferably about 0 to about 5 nm

Preferably, the solid surface is coupled with the electrical change detecting element for detecting the electrical change.

According to the invention, a free form of the electrically charged methylation detecting molecule is preferably removed before detecting the electrical change, thereby avoiding noise generated by the electrical charges carried by the free form of the electrically charged methylation detecting molecule. The manner of removal includes but is not limited to washing

In one preferred embodiment of the invention, the method is for detecting the position of the methylation of the target oligonucleotide molecule. By attaching the recognizing single-stranded oligonucleotide molecule on the solid surface, the electrical changes occurring in different positions of the duplex are able to be distinguished from each other. For example, when the electrically charged methylation detecting molecule binds to the methylated cytosine nucleotide positioned near the solid surface, a stronger electrical signal is detected by the electrical change detecting element coupled with the solid surface, because the electrical charges introduced by the electrically charged methylation detecting molecule are positioned near the electrical change detecting element. On the other hand, when the electrically charged methylation detecting molecule binds to the methylated cytosine nucleotide positioned away from the solid surface, a weaker electrical signal is detected by the electrical change detecting element coupled with the solid surface, because the electrical charges introduced by the electrically charged methylation detecting molecule are positioned away from the electrical change detecting element.

In one preferred embodiment of the invention, the method is for detecting the number or percentage of the methylation of the target oligonucleotide molecule. By attaching the recognizing single-stranded oligonucleotide molecule on the solid surface, the electrical changes occurring due to different numbers of methylated cytosine nucleotide of the duplex are able to be distinguished from each other. For example, when more electrically charged methylation detecting molecules bind to the target oligonucleotide molecule with more methylated cytosine nucleotides, a stronger electrical signal is detected by the electrical change detecting element coupled with the solid surface, because more electrical charges are introduced by the electrically charged methylation detecting molecule. On the other hand, when less electrically charged methylation detecting molecules bind to the target oligonucleotide molecule with less methylated cytosine nucleotides, a weaker electrical signal is detected by the electrical change detecting element coupled with the solid surface, because less electrical charges are introduced by the electrically charged methylation detecting molecule.

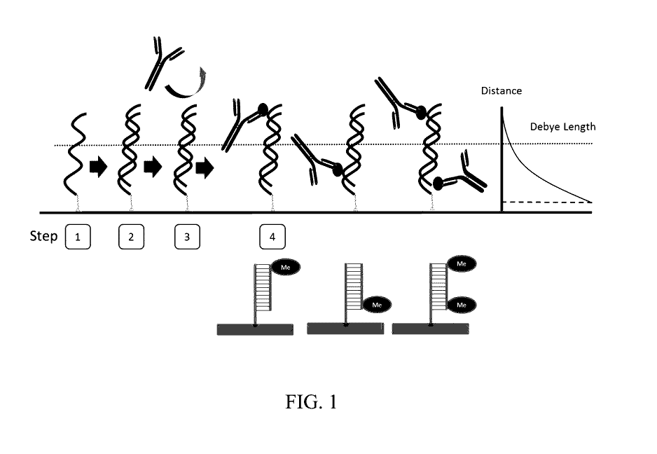

Referring to FIG. 1, a preferred embodiment of the invention is illustrated. In step 1, a recognizing single-stranded oligonucleotide molecule is attached to a solid surface. In step 2, a target oligonucleotide molecule is captured by the recognizing single-stranded oligonucleotide molecule to form a duplex according to base complementarity. In step 3, an electrically charged methylation detecting molecule is applied for contacting the duplex. In step 4, because the target oligonucleotide molecule comprises at least one methylated cytosine nucleotide, the electrically charged methylation detecting molecule binds to the least one methylated cytosine nucleotide, and an electrical change occurs due to the binding of an electrically charged methylation detecting molecule and the target oligonucleotide molecule.

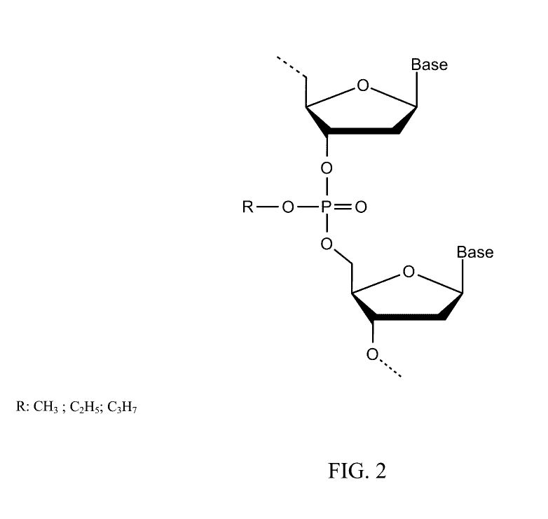

In one preferred embodiment of the invention, the recognizing single-stranded oligonucleotide molecule is a partially neutral single-stranded oligonucleotide comprising at least one electrically neutral nucleotide and at least one negatively charged nucleotide. The manner of rendering a nucleotide electrically neutral is not limited. In one embodiment of the invention, the electrically neutral nucleotide comprises a phosphate group substituted by an alkyl group. Preferably, the alkyl group is a C₁-C₆ alkyl group; more preferably, the alkyl group is a C₁-C₃ alkyl group. Examples of the C₁-C₃ alkyl group include but are not limited to methyl, ethyl and propyl. FIG. 2 shows one preferred embodiment of the electrically neutral nucleotide according to the invention. The negatively-charged oxygen atom in the phosphate group is changed to a neutral atom without charge. The way to substitute the phosphate group with the alkyl group can be applied according to common chemical reactions.

The negatively charged nucleotide according to the invention comprises a phosphate group with at least one negative charge. The unmodified nucleotide is preferably a naturally occurring nucleotide without modification or substitution. In one preferred embodiment of the invention, the negatively charged nucleotide comprises an unsubstituted phosphate group.

The partially neutral single-stranded oligonucleotide according to the invention is partially rendered electrically neutral. The sequence or length is not limited, and the sequence or length of the partially neutral single-stranded oligonucleotide can be designed according to a target oligonucleotide molecule based on the disclosure of the invention.

The numbers of electrically neutral nucleotides and negatively charged nucleotides depend on the sequence of the partially neutral single-stranded oligonucleotide and the condition under the duplex formation. The positions of the electrically neutral nucleotides and negatively charged nucleotides also depend on the sequence of the partially neutral single-stranded oligonucleotides and the condition under the duplex formation. The numbers and positions of the electrically neutral nucleotides and negatively charged nucleotides can be designed according to the available information based on the disclosure of the invention. For example, the number and position of the electrically neutral nucleotides can be designed by molecular modeling calculation based on double stranded (ds) structural energy, and the melting temperature (Tm) of dsDNA/DNA or dsDNA/RNA can then be determined by reference to the structural energy.

In one preferred embodiment of the invention, the partially neutral single-stranded oligonucleotide comprises a plurality of the electrically neutral nucleotides, and at least one negatively charged nucleotide is positioned between two of the electrically neutral nucleotides; more preferably, at least two negatively charged nucleotides are positioned between two of the electrically neutral nucleotides.

In one preferred embodiment of the invention, when the partially neutral single-stranded oligonucleotide according to the invention is applied, at least one electrically neutral nucleotide is positioned near the methylated cytosine nucleotide; more preferably, 2, 3, 4, or 5 electrically neutral nucleotides are positioned near the methylated cytosine nucleotide. In another aspect, at least one electrically neutral nucleotide is positioned at the downstream or upstream site of the methylated cytosine nucleotide; preferably, at least one electrically neutral nucleotide is positioned at the downstream and upstream sites of the methylated cytosine nucleotide.

By introducing the electrically neutral nucleotide, the melting temperature difference between perfect match double-stranded oligonucleotides and mismatched double-stranded oligonucleotides of the partially neutral single-stranded oligonucleotide according to the invention is higher compared with that of a conventional DNA probe. Without being restricted by theory, it is surmised that the electrostatic repulsion force between two strands is lowered by introducing the neutral oligonucleotide, and the melting temperature is raised thereby. By controlling the number and position of electrically neutral nucleotides, the melting temperature difference is adjusted to a desired point, providing a better working temperature or temperature range to differentiate the perfect and mismatched oligonucleotides, thereby improving capture specificity. Such design benefits consistency of the melting temperature of different partially neutral single-stranded oligonucleotides integrated in one chip or array. The number of reactions to be detected can be raised dramatically with high specificity and more detection units can be incorporated into a single detection system. The design provides better microarray operation conditions.

In one preferred embodiment of the invention, the partially neutral single-stranded oligonucleotide comprises a first portion attached to the solid surface; the length of the first portion is about 50% of the total length of the partially neutral single-stranded oligonucleotide; and the first portion comprises at least one electrically neutral nucleotide and at least one negatively charged nucleotide; more preferably, the length of the first portion is about 40% of the total length of the partially neutral single-stranded oligonucleotide; still more preferably, the length of the first portion is about 30% of the total length of the partially neutral single-stranded oligonucleotide.

In one preferred embodiment of the invention, the partially single-stranded nucleotide further comprises a second portion adjacent to the first portion. The second portion is located in the distal end to the solid surface. The second portion comprises at least one electrically neutral nucleotide and at least one negatively charged nucleotide. The description of the electrically neutral nucleotide and the negatively charged nucleotide is the same as that of the first portion and is not repeated herein.

In one preferred embodiment of the invention, a signal amplifier is further provided for enhancing the detection of the electrical change. The signal amplifier according to the invention preferably refers to a component that enhances the detection of the voltage change of the solid surface. For example, the signal amplifier can be nanogold particles attached to one end of the recognizing single-stranded oligonucleotide molecule.

In one preferred embodiment of the invention, the method is performed in a buffer with an ionic strength lower than about 50 mM; more preferably lower than about 40 mM, 30 mM, 20 mM or 10 mM. Without being restricted by theory, it is surmised that by applying the partially neutral single-stranded oligonucleotide, the duplex formed between the partially neutral single-stranded oligonucleotide with the target oligonucleotide molecule can happen without the need to suppress the electrostatic repulsive forces between the partially charged semi-neutral single-stranded oligonucleotide and its target. The hybridization is then driven by the base pairing and the stacking force of each strand. Consequently, the duplex can be formed at a lower salt condition. With FET, the lower ion strength increases the detection length (the debye length) and, in turn, enhances the detection sensitivity.

In one embodiment of the invention, the improved hybridization specificity for forming the duplex can be seen mainly in two aspects of FET detection compared to a conventional detection. First, the melting temperature difference is higher. Second, the buffer has a lower salt condition, and the FET detection length (the debye length) is increased. Both of these differences result in improvement of detection sensitivity.

In a preferred embodiment of the invention, several recognizing single-stranded oligonucleotide molecules are contained in one system to carry out several detections in one manipulation. For example, a plurality of recognizing single-stranded oligonucleotide molecules may be incorporated in a detection system. Preferably, the detection system is a microarray or a chip.

In one preferred embodiment of the invention, the method comprises steps of:
(a) providing
   the single strand of the target oligonucleotide molecule;
   the recognizing single-stranded oligonucleotide molecule;
   a reference single-stranded oligonucleotide molecule having the target sequence without methylated cytosine nucleotide; and
   the electrically charged methylation detecting molecule;
(b) capturing the single strand of the target oligonucleotide molecule with the recognizing single-stranded oligonucleotide molecule to form a target duplex, and capturing the reference single-stranded oligonucleotide molecule with the recognizing single-stranded oligonucleotide molecule to form a reference duplex, respectively;
(c) contacting the electrically charged methylation detecting molecule with the target duplex to form a target complex, and contacting the electrically charged methylation detecting molecule with the reference duplex, respectively; and removing a free form of the electrically charged methylation detecting molecule; and
(d) monitoring an electrical change between the target complex and the reference duplex.

As used herein, the term “a reference single-stranded oligonucleotide molecule” refers to an oligonucleotide molecule that has the target sequence without methylated cytosine nucleotide, providing a background with no methylation. Preferably, the reference single-stranded oligonucleotide molecule has the same structure as the target oligonucleotide molecule except the methylated cytosine nucleotides. In another aspect, the reference single-stranded oligonucleotide molecule and the target oligonucleotide molecule are both captured by the recognizing single-stranded oligonucleotide molecule, and the manipulations relating to the reference single-stranded oligonucleotide molecule and the target oligonucleotide molecule in steps (b) to (d) are performed simultaneously.

Preferably, the conditions for forming the target duplex and the reference duplex are the same. In another aspect, the conditions for forming the target complex and the reference complex are the same.

The electrical change between the target complex and the reference duplex represents the electrical change occurring due to the binding of the electrically charged methylation detecting molecule and the target oligonucleotide molecule.

The present invention is also to provide a kit for detecting methylation of a target oligonucleotide molecule comprising:
an electrically charged methylation detecting molecule having affinity to a methylated cytosine nucleotide; and
an electrical change detecting element for detecting an electrical change.

Preferably, the kit according to the invention further comprises the recognizing single-stranded oligonucleotide molecule as mentioned above.

Preferably, the kit according to the invention further comprises the signal amplifier as mentioned above.

Preferably, the kit according to the invention further comprises the reference single-stranded oligonucleotide molecule having the target sequence without methylated cytosine nucleotide as mentioned above.

The following examples are provided to aid those skilled in the art in practicing the present invention.

### EXAMPLES

### Synthesis of Partially Neutral Single-Stranded Oligonucleotide as Recognizing Single-Stranded Oligonucleotide Molecule:

Deoxy cytidine (n-ac) p-methoxy phosphoramidite, thymidine p-methoxy phosphoramidite, deoxy guanosine (n-ibu) p-methoxy phosphoramidite, and deoxy adenosine (n-bz) p-methoxy phosphoramidite (all purchased from ChemGenes Corporation, USA) were used to synthesize an oligonucleotide according to a given sequence based on solid-phase phosphotriester synthesis or by Applied Biosystems 3900 High Throughput DNA Synthesizer (provided by Genomics® Biosci & Tech or Mission Biotech).

The synthesized oligonucleotide was reacted with weak alkaline in toluene at room temperature for 24 hours, and the sample was subjected to ion-exchange chromatography to adjust the pH value to 7. After the sample was concentrated and dried, the partially neutral single-stranded oligonucleotide was obtained.

### Recognizing Single-Stranded Oligonucleotide Molecule Attachment:

Recognizing single-stranded oligonucleotide molecule attachment was performed by functionalization of the Si-nanowire (SiNW) surface layer (SiO₂). First, (3-Aminopropyl)triethoxysilane (APTES) was used to modify the surface. The silicon atom in the molecule of APTES performed a covalent bond with the oxygen of the hydroxyl group and converted the surface's silanol groups (SiOH) to amines.

Samples were immersed in 2% APTES (99% EtOH) for 30 minutes and then heated to 120° C. for 10 min. After this step was completed, amino groups (NH₂) were the terminal units from the surface.

Next, glutaraldehyde was used as a grafting agent for DNA attachment. Glutaraldehyde binding was achieved through its aldehyde group (COH) to ensure a covalent bond with the amino group of APTES. For this step, samples were immersed in 12.5% glutaraldehyde (10 mM sodium phosphate buffer) in liquid for 1 hour at room temperature.

The recognizing single-stranded oligonucleotide molecule was mixed with a bis-tris propane buffer at the concentration of 1 μM, and the modified SiNW surface was further immersed in the solution for 12 hours.

### Capturing Target Oligonucleotide Molecule

The sequences of the recognizing single-stranded oligonucleotide molecule (probe) and the target oligonucleotide molecule (target) are listed in Table 1.

TABLE 1

| | | SEQ ID |
|---|---|---|
| Sequence name | DNA sequence (5′→3′) | No. |
| SEPT9 DNA probe 1 | NH2-GCGCGCTGGCTGGGGC | 1 |
| | GCCGCGCCCGCGCT | |
| SEPT9 DNA target 1 | AGCGCGGGCGCGGCGCCCCA | 2 |
| | GCCAGCGCGC | |
| SEPT9 methyl DNA | AGCGCmeGGGCGCGGCGCCC | 3 |
| target 1 (C5) | CAGCCAGCGCGC | |
| SEPT9 methyl DNA | AGCGCGGGCGCGGCmeGCCC | 4 |
| target 2 (C14) | CAGCCAGCGCGC | |
| SEPT9 methyl DNA | AGCGCGGGCGCGGCGCCCCA | 5 |
| target 3 (C26) | GCCAGCmeGCGC | |
| SEPT9 DNA probe 2 | NH2-CGAAATGATCCCATCC | 6 |
| | AGCTGCGCGTTGACCGC | |
| SEPT9 DNA target 2 | GCGGTCAACGCGCAGCTGGA | 7 |
| | TGGGATCATTTCG | |
| SEPT9 methyl DNA | GCmeGGTCAACGCGCAGCTG | 8 |
| target 4 (C2) | GATGGGATCATTTCG | |
| SEPT9 methyl DNA | GCmeGGTCAACGCGCAGCme | 9 |
| target 5 (C2,16) | TGGATGGGATCATTTCG | |
| SEPT9 methyl DNA | GCmeGGTCAACGCGCAGCme | 10 |
| target 6 (C2,16,32) | TGGATGGGATCATTTCmeG | |
| SEPT9 methyl DNA | GCGGTCAACGCGCAGCTGGA | 11 |
| target 7 (C39) | TGGGATCATTTCGGACTTC | |
| | meGAAGGTGGG | |
| SEPT9 N-DNA probe | NH2-CnGAAnATGnATCnCC | 12 |
| | AnTCCnAGCTGCGCGTTGAC | |
| | CGC | |

### Cₘₑ: Methylated Cytosine Nucleotide

### Example 1

### Detecting the Position of the Methylation of the Target Oligonucleotide Molecule

The target oligonucleotide molecules were SEPT9 methyl DNA target 1 (C5), SEPT9 methyl DNA target 1 (C14), and SEPT9 methyl DNA target 1 (C26), having a methylated cytosine nucleotide at positions 5, 14, and 26, respectively.

The target oligonucleotide molecule was mixed with a bis-tris propane buffer at the concentration of 100 pM, and captured by SEPT9 DNA probe 1 attached to the SiNW for 30 minutes to form a duplex.

Anti-5mC antibody (0.25 μg/mL) as an electrically charged methylation detecting molecule was introduced to the duplex for reacting for 30 minutes.

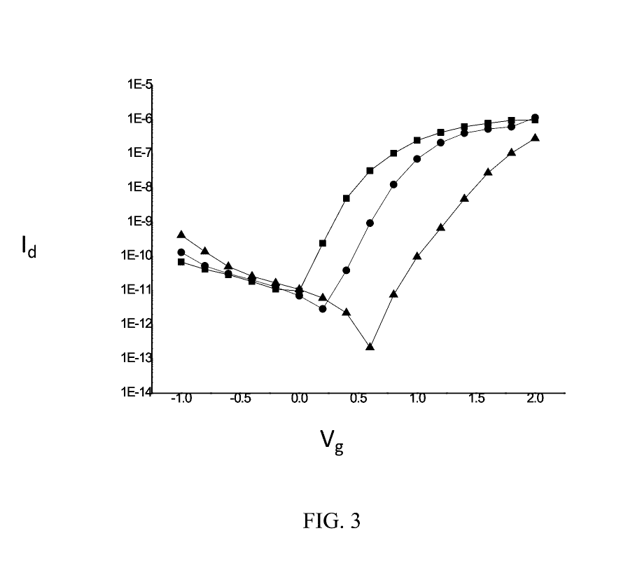

The results are shown in FIGS. 3 to 7.

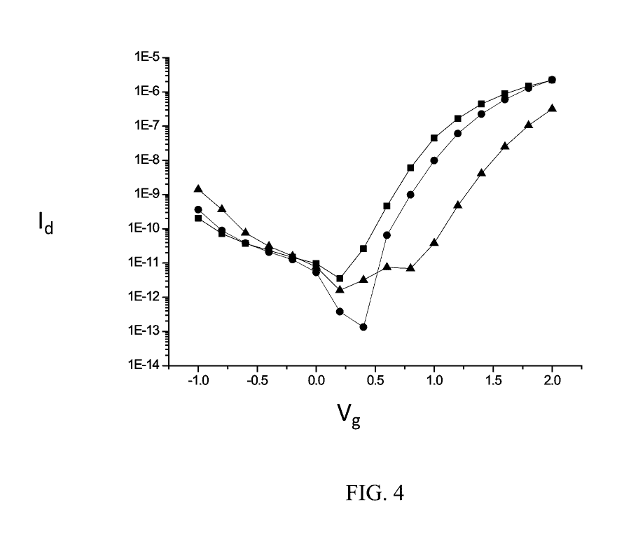

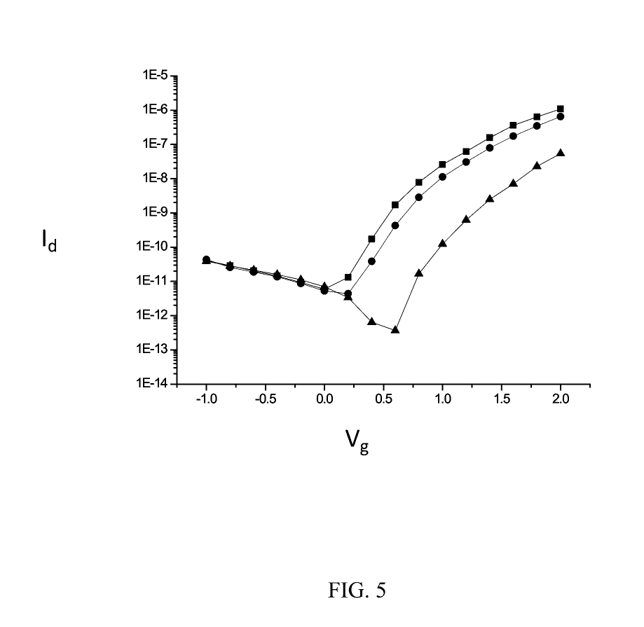

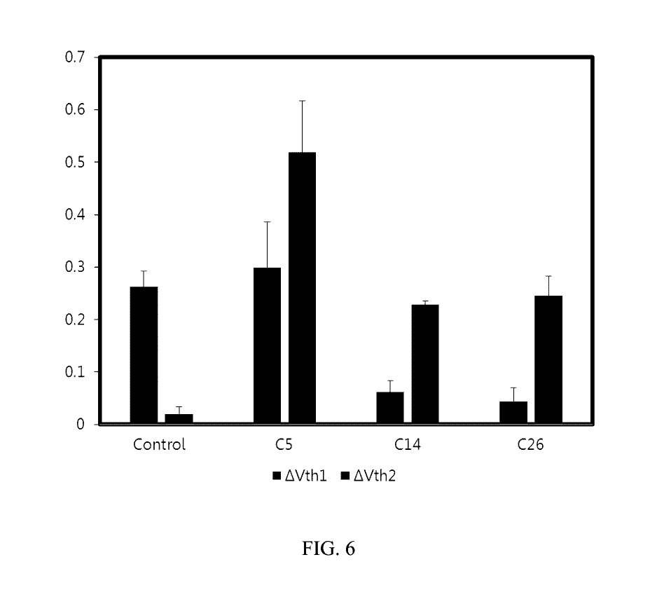

FIG. 3 shows the electrical signal of pSNWFET under analyte processing. “▪” line showed the I_{d}-V_{g} curve of pSNWFET processed with 10 mM, pH=7 bis-tris propane buffer. After the pSNWFET processed with the complementary DNA, the DNA compromised buffer was replaced with the same bis-tris propane buffer. Subsequently, electrical signal detection was processed and resulted as the “” line. Finally, buffer comprising the anti-methylcytosine antibody was injected and incubated on nanowire. Replacement of bis-tris propane buffer was followed by the incubation, and the electrical signal is shown as the “▴” line. Similar detections for different methylcytosine sites were performed and the results are shown in FIG. 4 and FIG. 5. The average AV_{S} induced by target DNA and anti-methylcytosine antibody is shown in FIG. 6. The following equation is used to normalize the electrical signal induced by antibody:

$$\frac{Δ {V}_{th} induced by antibody}{Δ {V}_{th} induced by target DNA}$$

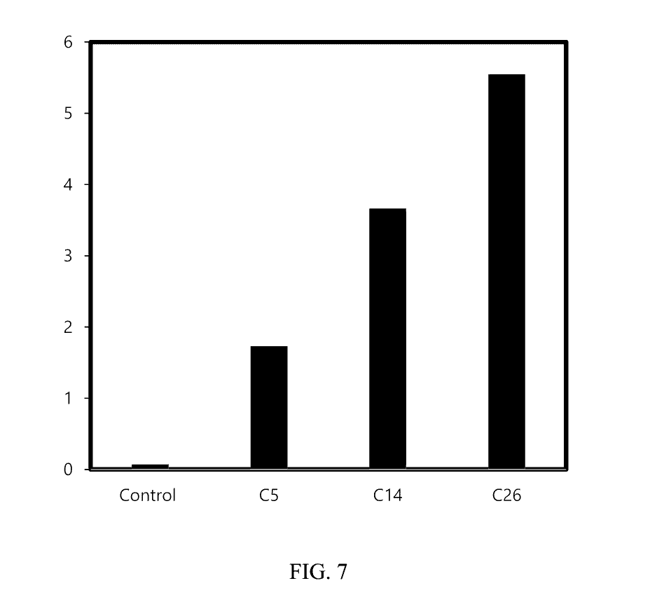

FIG. 7 shows the result of normalization—ratio of antibody to target DNA, which significantly demonstrates that the method according to the invention is for detecting the positions of the methylation of the target oligonucleotide molecule.

### Example 2

### Detecting the Number of the Methylated Cytosine Nucleotides in the Target Oligonucleotide Molecule

The target oligonucleotide molecules were SEPT9 methyl DNA target 4 (C2), SEPT9 methyl DNA target 5 (C2+C16), and SEPT9 methyl DNA target 6 (C2+C16+C32), having one, two or three methylated cytosine nucleotides, respectively.

The target oligonucleotide molecule was mixed with a bis-tris propane buffer at the concentration of 100 pM, and captured by SEPT9 DNA probe 1 attached to the SiNW for 30 minutes to form a duplex.

Anti-5mC antibody (0.25 μg/mL) as an electrically charged methylation detecting molecule was introduced to the duplex for reacting for 30 minutes.

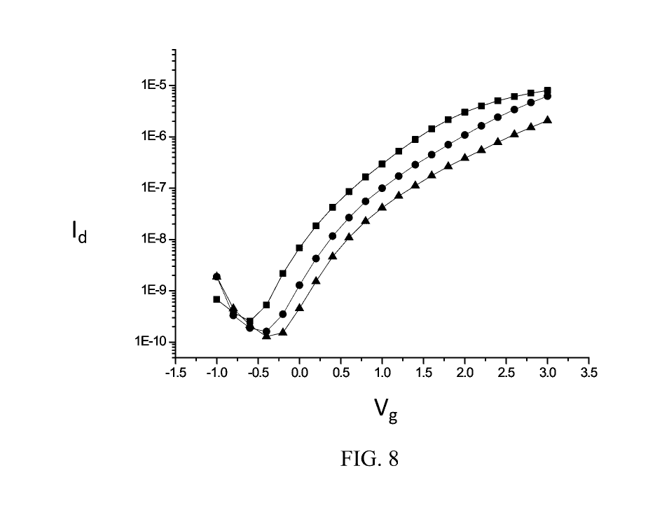

The results are shown in FIGS. 8 to 10.

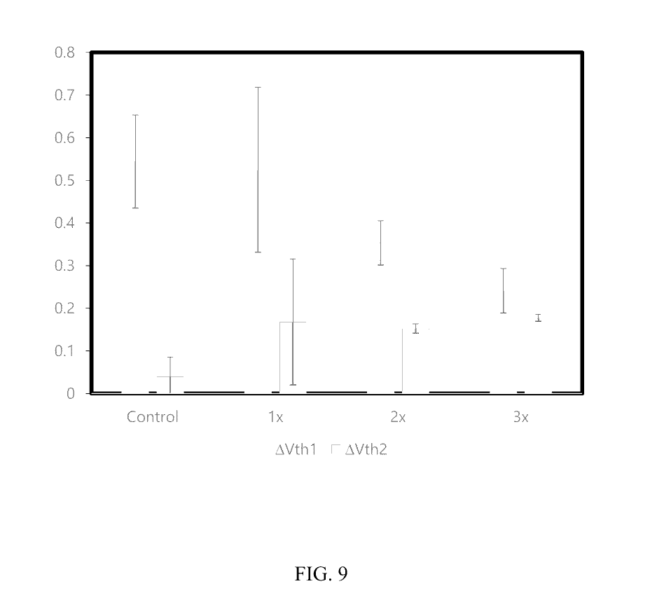

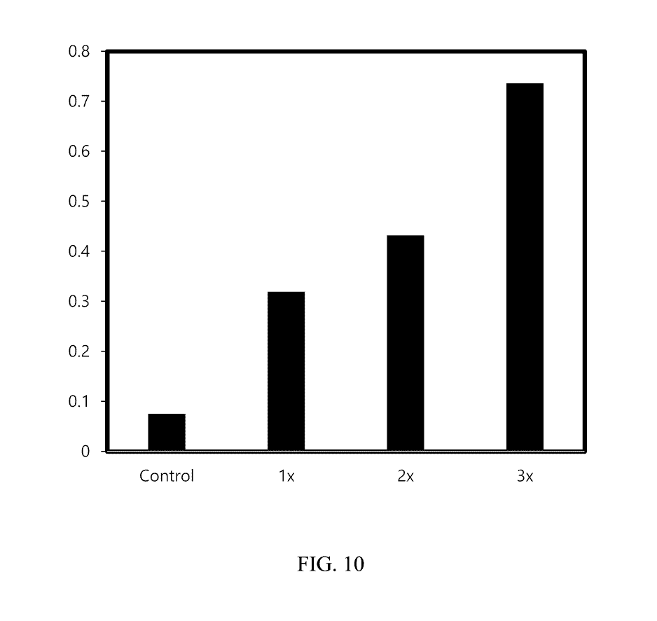

The target oligonucleotide molecules were synthesized with different numbers of methylcytosine, which provides different numbers of antibody binding sites. FIG. 8 shows the detection result of SEPT9 methyl DNA target 6. As the validation of distance effect, I_{d}-V_{g} curve was detected in the condition of bis-tris propane buffer, buffer containing DNA target and anti-methylcytosine antibody. FIG. 9 demonstrates the average of 3 times threshold voltage shift induced by the target DNA and antibody, respectively. FIG. 10 shows the normalization of threshold voltage shifts from FIG. 9, and it obviously indicates that the numbers of methylcytosine have positive correlation with the voltage shift.

### Example 3

### Detecting the Methylated Cytosine Nucleotides on a Single Strand Tail in the is Target Oligonucleotide Molecule

The target oligonucleotide molecule was SEPT9 methyl DNA target 7 (C39).

The target oligonucleotide molecule was mixed with a bis-tris propane buffer at the concentration of 100 pM, and captured by SEPT9 DNA probe 2 attached to the SiNW for 30 minutes to form a duplex.

Anti-5mC antibody (1 ng/mL) as an electrically charged methylation detecting molecule was introduced to the duplex for reacting for 30 minutes.

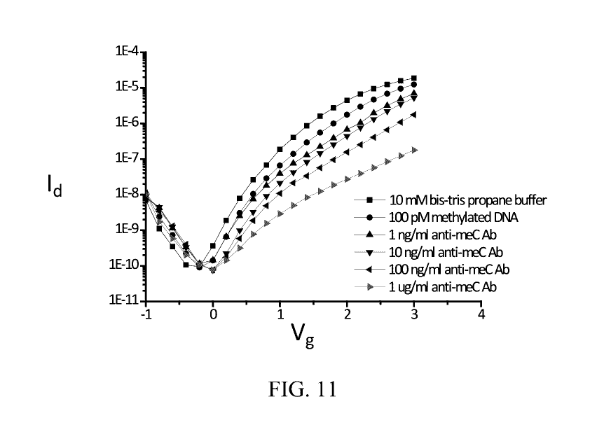

The results are shown in FIG. 11. The figure demonstrates that the methylated cytosine positioned on a single strand tail of the duplex formed by the SEPT9 DNA probe 2 with SEPT9 methyl DNA target 7 increases the detection limit to 1 ng/ml anti-methylcytosine antibody.

### Example 4

### Detecting the Methylated Cytosine Nucleotides in the Target Oligonucleotide Molecule with N-DNA

The target oligonucleotide molecule was SEPT9 methyl DNA target 6 (C2,16,32).

The target oligonucleotide molecule was mixed with a bis-tris propane buffer at the concentration of 100 pM, and captured by SEPT9 N-DNA probe attached to the SiNW for 30 minutes to form a duplex.

Anti-5mC antibody (0.1 μg/mL) as an electrically charged methylation detecting molecule was introduced to the duplex for reacting for 30 minutes.

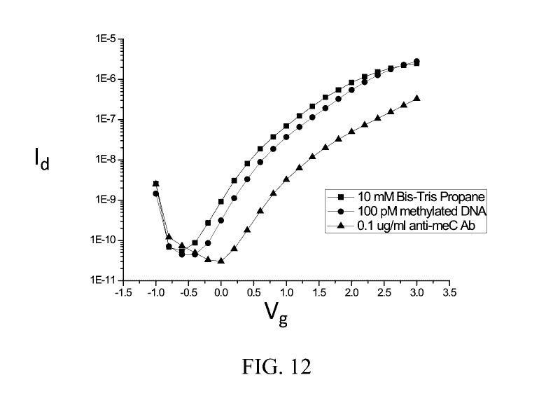

The results are shown in FIG. 12. The figure demonstrates that the methylated cytosine positioned on a single strand tail of the duplex formed by the SEPT9 DNA probe 2 with SEPT9 methyl DNA target 7 increases the detection limit to 1 ng/ml anti-methylcytosine antibody.

FIG. 12 shows I_{d}-V_{g} curve of partially neutral single-stranded oligonucleotide (N-DNA) probe binding with SEPT9 methyl DNA target 6 (C2, C16, C32) and antibody. Compared to FIG. 8, it shows that N-DNA has the improved effect in sensitivity.

While the present invention has been described in conjunction with the specific embodiments set forth above, many alternatives thereto and modifications and variations thereof will be apparent to those of ordinary skill in the art. All such alternatives, modifications and variations are regarded as falling within the scope of the present invention.

What is claimed is:

1. A method for detecting methylation of a target oligonucleotide molecule, comprising obtaining an electrical change occurring due to the binding of an electrically charged methylation detecting molecule and the target oligonucleotide molecule; wherein the electrically charged methylation detecting molecule has affinity to a methylated cytosine nucleotide.

2. The method according to claim 1, wherein the electrically charged methylation detecting molecule is selected from the group consisting of an anti-methylcytosine antibody, a methyl binding domain protein and a restriction enzyme.

3. The method according to claim 1, wherein the electrical change is a threshold voltage shift change.

4. The method according to claim 1, wherein the target oligonucleotide molecule has a target sequence with at least one methylated cytosine nucleotide, and the method comprises:
capturing a single strand of the target oligonucleotide molecule by a recognizing single-stranded oligonucleotide molecule to form a duplex according to base complementarity;
providing the electrically charged methylation detecting molecule; and
binding the duplex with the electrically charged methylation detecting molecule and detecting the electrical change due to the binding.

5. The method according to claim 4, wherein the duplex formed by the single strand of the target oligonucleotide molecule and the recognizing single-stranded oligonucleotide molecule comprises a bulge and the methylated cytosine nucleotide is disposed in the bulge.

6. The method according to claim 4, wherein the recognizing single-stranded oligonucleotide molecule is attached to a solid surface or the recognizing single-stranded oligonucleotide molecule is spaced apart from the solid surface by a distance.

7. The method according to claim 6, wherein the solid surface is a transistor surface of a field-effect transistor (FET) or a metal surface of a surface plasmon resonance (SPR).

8. The method according to claim 6, wherein the material of the solid surface is polycrystalline silicon or single crystalline silicon.

9. The method according to claim 6, wherein the solid surface is coupled with an electrical change detecting element for detecting the electrical change.

10. The method according to claim 9, wherein the electrical change detecting element is a field-effect transistor or a surface plasmon resonance.

11. The method according to claim 4, wherein the recognizing single-stranded oligonucleotide molecule is a partially neutral single-stranded oligonucleotide comprising at least one electrically neutral nucleotide and at least one negatively charged nucleotide.

12. The method according to claim 11, wherein the electrically neutral nucleotide comprises a phosphate group substituted by a C₁-C₆ alkyl group.

13. The method according to claim 11, wherein the negatively charged nucleotide comprises an unsubstituted phosphate group.

14. The method according to claim 11, wherein the recognizing single-stranded oligonucleotide molecule is attached to a solid surface, and the partially neutral single-stranded oligonucleotide comprises a first portion attached to the solid surface; the length of the first portion is about 50% of the total length of the partially neutral single-stranded oligonucleotide; and the first portion comprises at least one electrically neutral nucleotide and at least one negatively charged nucleotide.

15. The method according to claim 1, comprising steps of:
(a) providing
the single strand of the target oligonucleotide molecule;
the recognizing single-stranded oligonucleotide molecule;
a reference single-stranded oligonucleotide molecule having the target sequence without methylated cytosine nucleotide; and
the electrically charged methylation detecting molecule;
(b) capturing the single strand of the target oligonucleotide molecule with the recognizing single-stranded oligonucleotide molecule to form a target duplex, and capturing the reference single-stranded oligonucleotide molecule with the recognizing single-stranded oligonucleotide molecule to form a reference duplex, respectively;
(c) contacting the electrically charged methylation detecting molecule with the target duplex to form a target complex, and contacting the electrically charged methylation detecting molecule with the reference duplex, respectively; and removing a free form of the electrically charged methylation detecting molecule; and
(d) monitoring an electrical change between the target complex and the reference duplex.

16. A kit for detecting methylation of a target oligonucleotide molecule comprising:
an electrically charged methylation detecting molecule having affinity to a methylated cytosine nucleotide; and
an electrical change detecting element for detecting an electrical change.

17. The kit according to claim 16, wherein the electrically charged methylation detecting molecule is selected from the group consisting of an anti-methylcytosine antibody, a methyl binding domain protein and a restriction enzyme.

18. The kit according to claim 16, which further comprises a recognizing single-stranded oligonucleotide molecule which is able to capture a single strand of the target oligonucleotide molecule to form a duplex according to base complementarity.

19. The kit according to claim 18, wherein the recognizing single-stranded oligonucleotide molecule is able to form a duplex comprising a bulge with the single strand of the target oligonucleotide molecule, and the methylated cytosine nucleotide is disposed in the bulge.

20. The kit according to claim 19, wherein the recognizing single-stranded oligonucleotide molecule is attached to a solid surface or the recognizing single-stranded oligonucleotide molecule is spaced apart from the solid surface by a distance.

21. The kit according to claim 20, wherein the solid surface is a transistor surface of a field-effect transistor or a metal surface of a surface plasmon resonance.

22. The kit according to claim 20, wherein the material of the solid surface is polycrystalline silicon or single crystalline silicon.

23. The kit according to claim 16, wherein the electrical change detecting element is a field-effect transistor or a surface plasmon resonance.

24. The kit according to claim 17, wherein the recognizing single-stranded DNA molecule is a partially neutral single-stranded oligonucleotide comprising at least one electrically neutral nucleotide and at least one negatively charged nucleotide.

25. The kit according to claim 24, wherein the electrically neutral nucleotide comprises a phosphate group substituted by a C₁-C₆ alkyl group.

26. The kit according to claim 24, wherein the negatively charged nucleotide comprises an unsubstituted phosphate group.

27. The kit according to claim 24, wherein the recognizing single-stranded oligonucleotide molecule is attached to a solid surface, and the partially neutral single-stranded oligonucleotide comprises a first portion attached to the solid surface; the length of the first portion is about 50% of the total length of the partially neutral single-stranded oligonucleotide; and the first portion comprises at least one electrically neutral nucleotide and at least one negatively charged nucleotide.

28. The kit according to claim 16 further comprising a reference single-stranded oligonucleotide molecule having the target sequence without methylated cytosine nucleotide.
